(19) United States
(12) Patent Application Publication
(10) Pub. No.: US 2021/0315927 A1
(43) Pub. Date: Oct. 14, 2021

(54) TREATING ARDS USING A POLY-OXYGENATED ALUMINUM HYDROXIDE

(71) Applicant: HEMOTEK LLC, PLANO, TX (US)

(72) Inventors: John W. Woodmansee, JR., Frisco, TX (US); Brooks Bash, Plano, TX (US)

(21) Appl. No.: 17/223,867

(22) Filed: Apr. 6, 2021

Related U.S. Application Data
(60) Provisional application No. 63/007,119, filed on Apr. 8, 2020.

(51) Int. Cl. A61K 33/08

(52) U.S. Cl. CPC A61K 33/08

(57) ABSTRACT

A method of administering a poly-oxygenated aluminum hydroxide containing free oxygen gas molecules (O₂) to prevent ARDS, and reduce the effects of ARDS in mammals. The poly-oxygenated aluminum hydroxide is marketed as Ox66™ by Hemotek, LLC of Plano, Tex., and is effective to increase oxygenation of a mammal suffering from ARDS, and also to increase a P_{A}O₂/FiO₂ ratio value.

### CLAIM OF PRIORITY

This application claims priority of U.S. Provisional Patent Application U.S. Ser. No. 63/007,119 entitled TREATING A VIRUS USING A POLY-OXYGENATED ALUMINUM HYDROXIDE filed Apr. 8, 2020, the teachings of which are incorporated herein by reference in their entirety.

### TECHNICAL FIELD

This disclosure is directed to treating ARDS in a mammal.

### BACKGROUND

Acute respiratory distress syndrome (ARDS) is a failure of pulmonary gas exchange resulting from traumatic injury or pathology. It has received widespread attention in the last year as a primary driver of mortality during COVID-19 infection. ARDS can be caused by numerous respiratory ailments, such as chronic obstructive pulmonary disease (COPD). Current approaches to improving oxygenation (e.g., ventilation and supplemental oxygen) are, by nature, limited in use because they are forced through already dysfunctional lungs causing additional damage.

Though typically characterized as an affliction of the lungs, the mechanisms underlying the onset and malignancy of ARDS span multiple organ systems. If uncontrolled, the widespread and prolonged hypoxia induced by ARDS degrades the barrier function of the gut, releasing the ordinarily beneficial population of the gut microbiome into the body, causing systemic inflammation, hypotension, and hypoperfusion due to the perceived infection. This further reduces oxygen delivery to critical organs, giving rise to multi-organ failure, which is the primary cause of death.

### SUMMARY

A method of administering a poly-oxygenated aluminum hydroxide containing free oxygen gas molecules (O₂) to prevent ARDS, and reduce the effects of ARDS in mammals. The poly-oxygenated aluminum hydroxide is marketed as Ox66™ by Hemotek, LLC of Plano, Tex., and is effective to increase oxygenation of a mammal suffering from ARDS, and also to increase a P_{A}O₂/FiO₂ ratio value.

### BRIEF DESCRIPTION OF THE DRAWINGS

FIG. 1 illustrates a chart of the P_{A}O₂/FiO₂ ratio value for different groups of mammals;

FIG. 2 illustrates a chart of vital signs of mammals upon the induction of ARDS, sorted by mammal groups;

FIG. 3 illustrates a graph of the ISF PO₂ data of FIG. 2;

FIG. 4 illustrates a graph of the mean arterial pressure based on the data of FIG. 2;

FIG. 5 illustrates data of arterial blood samples for each group collected at 60 minutes post-induction; and

FIG. 6 illustrates the survival of the animals in all three groups during the study.

### DETAILED DESCRIPTION

This disclosure is directed to an oxygenating therapeutic to prevent ARDS in mammals, and to reduce the effects of ARDS in mammals using a poly-oxygenated aluminum hydroxide comprising a clathrate containing oxygen gas O₂ molecules, such as Ox66™ manufactured by and available from Hemotek LLC of Plano, Tex., the assignee of this application. Ox66™ is a poly-oxygenated aluminum hydroxide composed of approximately 66.2% oxygen and organized as a true clathrate, allowing for the capture of oxygen molecules within its lattice structure. In an example, the Ox66™ is delivered by injection or IV.

The following description includes a study of mechanically-induced ARDS in mammals, including examples and information enables a person skilled in the art to make and use the subject matter set forth in the appended claims, but may omit certain details already well-known in the art. The following detailed description is, therefore, to be taken as illustrative and not limiting.

Standard clinical treatments for ARDS in mammals include lung-protective (low tidal volume) ventilation, supplemental oxygen, prone positioning, and extracorporeal membrane oxygenation. increased physiologic dead space ventilation (VD/VT) is the hallmark of ARDS and a primary indicator of mortality. It limits the efficacy of interventions using the respiratory pathway and can introduce additional complications arising from the intervention itself, such as ventilation-induced alveolar overdistention. The crippled gas exchange dynamics of the ARDS-affected lung poses a further challenge to treatment using supplemental oxygen, which requires dynamic, titrated dosing based on patient status and selected ventilation parameters.

The Berlin criteria serves as the current international consensus definition of ARDS in adults, defining the disorder as a P_{A}O₂/FiO₂ ratio value (partial pressure of O₂ in the arteries over fraction of inspired O₂ in the inspired air) of less than 300 mmHg. This 300 mmHg threshold is employed clinically for both identification of ARDS and adjustment of treatment in accordance with current patient status.

This disclosure uses a model of mechanically-induced ARDS to investigate the oxygen carrying ability of a novel, injectable solid-state oxygen carrying compound, namely, Ox66™, and the effect of administering Ox66™ to reduce the effects of ARDS in mammals.

### Animals

The following protocol and experimental procedures were approved by the Thomas D Morris, Inc. Institutional Animal Care and Use Committee (Protocol #20-007) and are consistent with the National Institutes of Health guidelines for the humane treatment of laboratory animals. Study subjects were male Sprague Dawley rats (N=21; 250-300 g; 8-10 weeks old; Charles River Laboratories, Inc., Wilmington, Mass. housed in pairs with a 12/12 day night cycle and provided ad libitum access to feed and water.

### Surgical Preparation

Animals were inducted with 1-5% isoflurane in air for initial preoperative preparation and cannulations. The anesthetic plane was maintained with a continuous intravenous infusion (0.1 mg/kg/min) into a femoral vein using a pre-filled syringe of alfaxalone acetate (Alfaxan, Schering-Plough Animal Health, Welwyn Garden City, UK) that was connected to a syringe pump (Genie Touch™, Kent Scientific, Torrington, Conn.). A second femoral vein was cannulated to be used for infusion of IV treatments. A femoral artery cannula was connected to a pressure transducer for continuous monitoring of arterial blood pressures (BIOPAC MP-150, BIOPAC Systems, Goleta, Calif.). The same femoral artery cannula was also used to collect blood samples for analysis of blood gases and chemistry (ABL90 Flex, Radiometer, Denmark). Cannulas were kept free of clots with heparinized phosphate-buffered saline (20 IU Heparin Sodium per ml; Pfizer, New York, N.Y.), which was not infused into the animal. A tracheal tube was inserted to maintain airway patency, and animals continued to inspire room air without artificial ventilation until baseline metrics had been collected. Next, animals were mechanically ventilated (RoVent® Jr, Kent Scientific, Torrington, Conn.) with medical air (21% oxygen, 78% nitrogen: Airgas, Radnor Township, Pa.) for the remainder of the study.

The rat spinotrapezius muscle was utilized for the investigation of microcirculatory parameters. This thin skeletal muscle was exteriorized and secured in situ onto a thermostable animal platform adapted for microcirculatory studies and measurement of the partial pressure of oxygen in the interstitium (ISF PO₂). After the experiment, rats were euthanized with an IV infusion of Euthasol (150 mg/kg, pentobarbital component; Delmarva, Midlothian, Va.), while under anesthesia.

### Intravital Microscopy

Observation and measurement of the exteriorized spinotrapezins preparation were carried out with an upright microscope (Axioimager2m, Carl Zeiss AG, Oberkochen, Germany) configured for trans-illumination through a 20X/0.8 objective (Plan-APOCHROMATE, Zeiss) modified for phosphorescence quenching microscopy. Trans-illumination was used to select measurement sites, establish appropriate focal planes, and verify flow conditions.

### Phosphorescence Quenching Microscopy

Measurements of interstitial oxygen tension (ISF PO₂) in the exteriorized spinotrapezius muscle and the technical setup have teen previously described in depth. Briefly, the phosphor (Oxyphore R0; Frontier Scientific, Newark, Del.) was topically applied to the tissue and allowed to diffuse into the interstitium. The phosphor probe was excited by a green laser diode operating a 520 nm (NDG7475 1W; Nichia Tokushima, Japan) in an octagonal region 300 μm in diameter at a frequency of 1 Hz. The excitation light pulse was passed through a filter cube consisting of a narrow-band filter (525 CWL Narrowband, Edmund Optics, Barrington, N.J.), a dichroic mirror (567 nm DMLP Longpass, Thorlabs, Newton, N.J.), and a wide-band filter (Longpass Cut-on >650 nm, Thorlabs, Newton, N.J.) for selective collection of phosphorescence emission. The phosphorescence signal was collected by a photomultiplier tube (R9110, Hamamatsu) and routed through a custom-built signal processor, collected by a data acquisition device (NI PCIe-6361, National Instruments, Austin Tex.) and stored digitally on a computer. Decay curves were fitted to a rectangular distribution model (17) and translated to discrete measurements of ISF PO₂.

### Ox66™ Preparation

Roughly 25 mg of Ox66™ was suspended in 1.5 mL of normal saline (BD PosiFlush™, Becton, Dickinson and Company, Franklin Lakes, N.J.) and allowed to settle for 3 minutes. Precipitation of larger particles by gravity formed a pellet at the bottom of the tube, and 1 mL of the supernatant was drawn for use as a single treatment dose. This process was repeated in full for each subsequent dose administered.

### Treatment Groups

Rats were randomly assigned to receive either an IV infusion Ox66™ prepared as described above, an infusion of 2.5 mL normal saline (vehicle control), or no treatment (sham). IV infusions were delivered through the femoral vein cannula. Three treatment doses were given at 5 minute intervals for the first 10 minutes.

### Protocol

Following surgical preparation, animals were allowed to reach a steady state (15 to 30 min) on the 3D printed thermoregulated platform used for intravital microscopy. Baseline (BL) measurements—spontaneous breathing at 100% tidal volume—consisted of the following systemic variables: mean arterial pressure (MAP), pulse pressure (PP), heart rate (HR), hindlimb oximetry, and temperature. Microcirculatory data was collected from three to five interstitial sites randomly selected for ISF PO₂ per experiment. Additionally, 65 mL of arterial blood was collected for oximetry and chemical analysis via ABL90 Flex (Radiometer; Copenhagen, Denmark). Systemic and microcirculatory metrics were recorded every 15 minutes, and blood gases were recorded at BL and 60, 120, 180 minutes.

Once BL metrics were collected, the first treatment dose of saline or Ox66™ was administered and ARDS was immediately introduced to the intubated animals by connecting the intubated animals to a mechanical ventilator (RoVent® Jr), calibrated to maintain a 50% tidal volume reduction relative to baseline. The RoVent® Jr came with presets for both breath rate and tidal volume based on a rodent's weight. Thus, for a 300 g rat whose preset tidal volume was 2.19 mL, the tidal volume was reduced to 1.095 mL. The reduced tidal volume remained for the entirety of the experiment. The second and third treatment doses were given at 5 minutes and 10 minutes of ARDS respectively.

### Statistics

Data are expressed as mean± standard error of the mean (SEM). Statistical comparisons within and between experimental groups were made using two-way ANOVA (repeated measures or multiple comparisons as appropriate; Prism 8, GraphPad Software Inc., San Diego, Calif.). Significance was defined as p<0.05. Intragroup comparisons were to baseline, while intergroup comparisons were at each appropriate time point. In cases where a significant difference was detected, a high stringency multiple comparison test (Tukey's or Sidak's) was performed. An unpaired T-test was used for single metrics (weight, age, etc).

### Animal Characteristics

Animal weights (Ox66™: 289±6; Saline: 287±5; Sham: 289±5, p=0.874) were equivalent between groups. All assessed metrics were also equivalent at baseline with the exception of heart rate (Ox66™ vs Sham, p<0.05) and pulse pressure (Ox66™ vs Vehicle, p<0.05).

### P_{A}O₂/FiO₂ Ratio

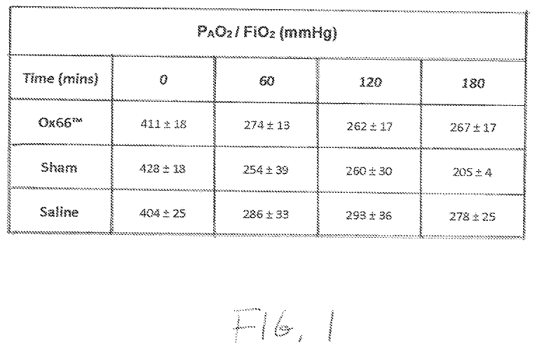

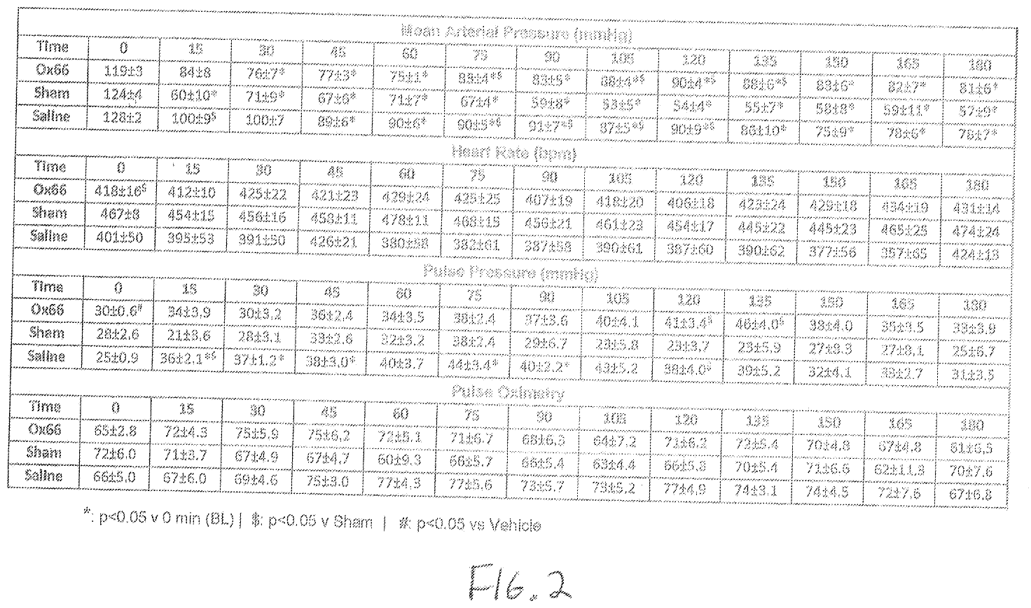

The PAO2/FiO2 ratio for each treatment group was calculated but not analyzed for statistical significance and is presented in FIG. 1. Ox66™ treated animals and sham animals (untreated animals) started the study at a P_{A}O₂/FiO₂ ratio value of 411 and 428 mmHg, respectively. Although Ox66™ treated animals had decreased P_{A}O₂/FiO₂ ratio values into the ARDS territory (below 300 mmHg) following the onset of the mechanically-induced ARDS, the values were “maintained” in the high 200s (274, 262 and 267 for the 1^{st}, 2^{nd} and 3^{rd} hour). Sham animals demonstrated a poorer outcome, where the values approached a more severe form of ARDS by the end of the study (254, 260 and 205 for the 1^{st}, 2^{nd} and 3^{rd} hour).

### Interstitial Fluid PO₂ in the Spinotrapezius

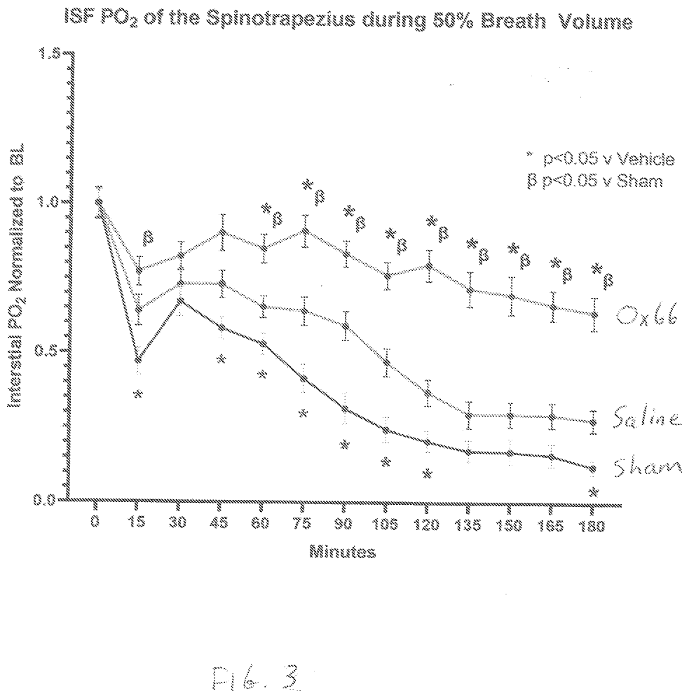

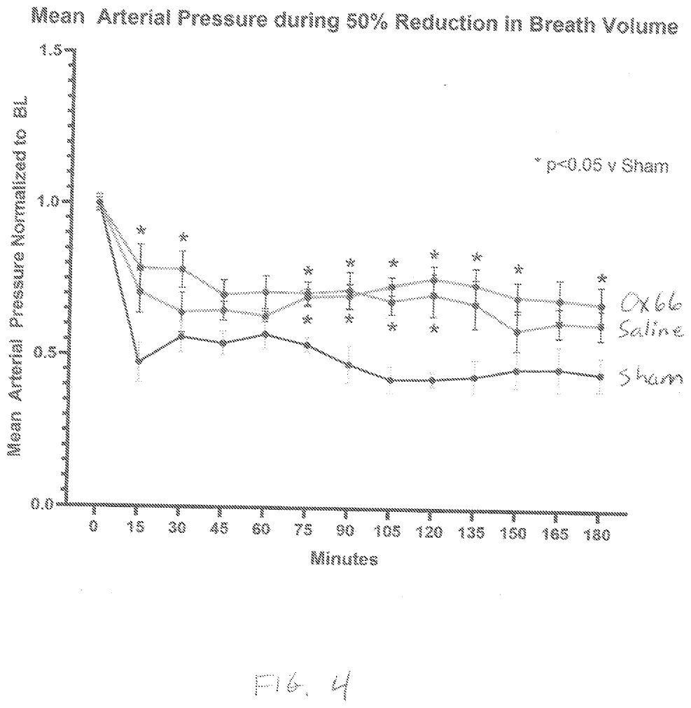

As shown in FIG. 2 and FIG. 3, the induction of ARDS by 50% tidal volume reduction caused an immediate drop in interstitial fluid PO₂ (ISF PO₂) in all groups, though the sham group showed a significantly greater reduction than both the Ox66™ group and the saline group. FIG. 3 is a graphical representation of the PO₂ data of FIG. 2. Ox66™ animals had the highest average ISF PO₂ values throughout the entire study, but only the comparison to the sham group yielded significance at this time point (t=15 min, p<0.05). All groups showed a gradual rise in ISF PO₂ following the initial drop, general reaching a peak between 30 and 45 minutes. Following this, PO₂ plateaued or steadily declined in each group until study termination. The difference in ISF PO₂ between Ox66™ and both control groups became significant at 60 minutes post-induction (p<0.05) and remained significant until the study's end. Saline-treated animals showed significantly greater ISF PO₂ values than sham group animals between 45 and 120 minutes, despite remaining significantly lower than the Ox66™ group.

### Systemic Cardiovascular Metrics

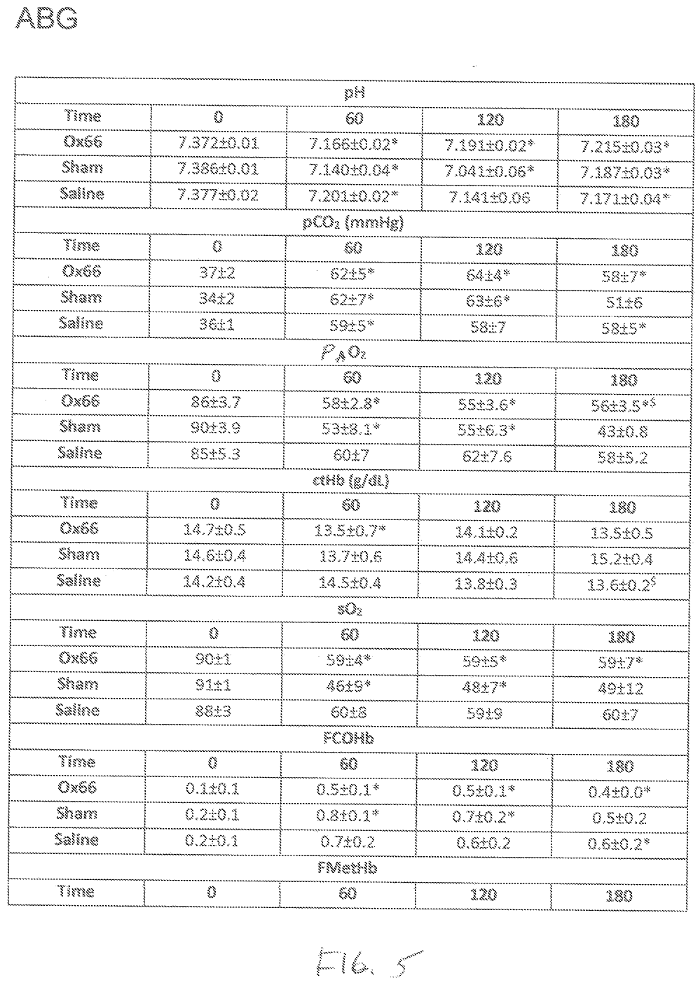

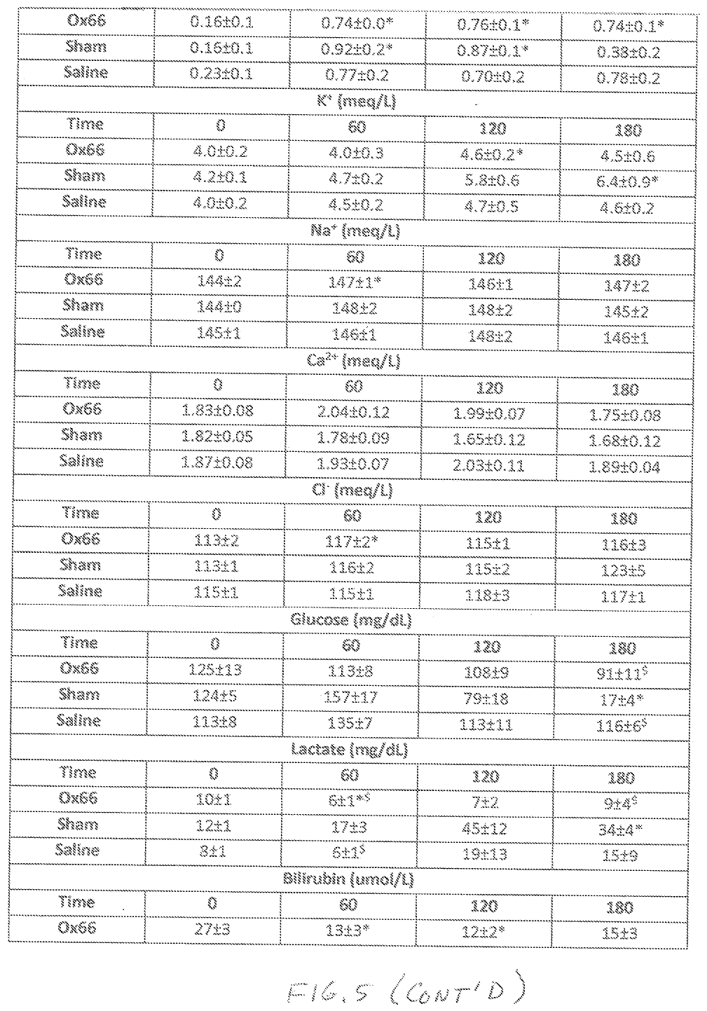

As shown in FIG. 4, the induction of ARDS to the mammal caused a decrease in mean arterial pressure (MAP) across all three groups, with the reduction generally becoming significant between 15 and 45 minutes (p<0.05). FIG. 4 is a graph of the MAP data of FIG. 2. A significant difference in pulse pressure between Ox66™ group and saline group was detected only at baseline, and the two groups showed no significant differences in MAP, heart rate, or pulse pressure at any other time points. Both the Ox66™ group and saline group showed significantly greater MAP than the sham group between 75 and 120 minutes, with the Ox66™ group maintaining a significant elevation until 150 minutes. There was no difference in pulse oximetry between groups or with respect to baseline at any point.

### Arterial Blood Gases (ABG) and Blood Chemistry

Intravenous Ox66™ was evaluated against saline and sham in a model of mechanically-induced ARDS in rodents. Arterial blood chemistry was assessed hourly and tissue PO₂ in the peripheral microcirculation, which is the earliest site of cardiovascular response to injury, was regularly measured using phosphorescence quenching microscopy. Groups of animals treated with Ox66™ exhibited higher interstitial fluid PO₂ (ISF PO₂) than the sham group or the saline group treated animals at every time point following baseline, as well as lower plasma lactate values than those measured at baseline despite the induced respiratory challenge.

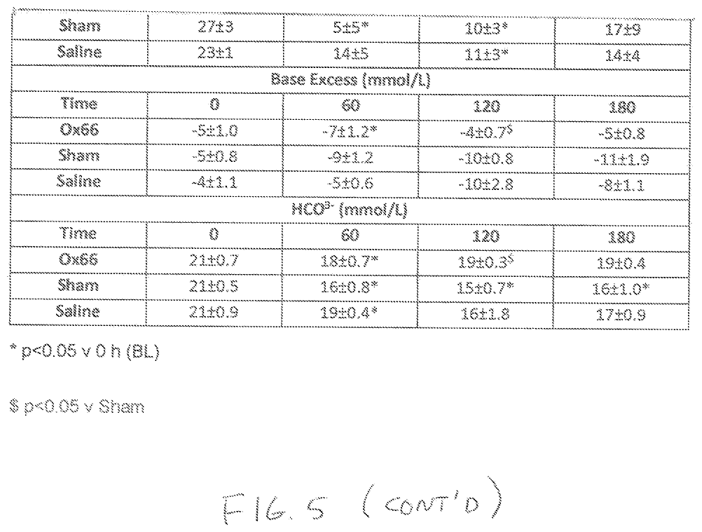

FIG. 5 illustrates arterial blood samples for each group at baseline and every 60 minutes post-induction of ARDS. Compared to sham, Ox66™ treated animals were less acidotic (higher pH), had a higher P_{A}O₂, improved oxygen saturation (sO₂), and lower amounts of lactate. This strongly supports the oxygen delivery mechanism of Ox66™ since increased bioavailable oxygen increases the P_{A}O₂ and sO₂, and lowers the amount of lactate (lactic acid), which is a by-product of glycolysis (anerobic respiration). Because there is less lactic acid associated with Ox66™ treated animals, they are also less acidic. Hence, the pH in Ox66™ treated animals is higher than sham animals.

The current in vivo model utilized a mechanically-induced ARDS model that investigates the early part of the injury. Though ARDS is typically characterized as an affliction of the lungs, the mechanisms underlying the onset and malignancy of ARDS span multiple organ systems. If uncontrolled, the widespread and prolonged hypoxia induced by ARDS degrades the barrier function of the gut, releasing the ordinarily beneficial population of the gut microbiome into the body, causing systemic inflammation, hypotension, and hypoperfusion due to the perceived infection. This further reduces oxygen delivery to critical organs, giving rise to multi-organ failure, which is the primary cause of death. Thus, it is conceivable that the correction of physiological hypoxia through Ox66™ can mitigate the inflammatory responses associated with severe ARDS.

Precise dosing of treatment in the Ox66™ group was not determined due to the use of the supernatant for treatment rather than the raw suspension of the product. However, taking the weight difference in the dry product before/after treatment showed that each animal cumulatively received 1-3 mg of product across the three doses administered. The results obtained from what is essentially a single bolus of treatment in this study show that Ox66™ has potent and fast-acting anti-hypoxic effects that can last well after delivery of the drug has ceased.

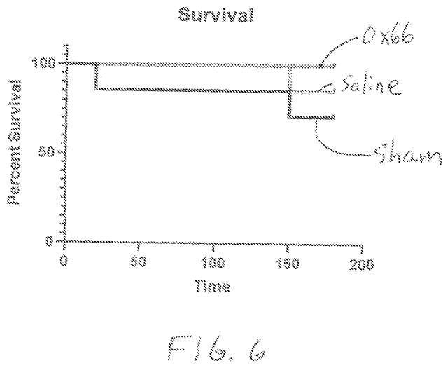

FIG. 6 illustrates the survival of the animals in all three groups during the study. All Ox66™ treated animals survived the study's conclusion, one saline and two sham animals did not.

### Significance

While animals treated with Ox66™ showed significantly improved tissue oxygenation relative to controls, they did not show supraphysiologic levels of P_{A}O₂ or sO₂ as is typically seen in when ARDS is treated with supplemental oxygen, as is currently standard. In addition, because Ox66™ can be administered by IV and injection, it carries no risk of hyperoxic acute lung injury or ventilator acute lung injury, both of which carry significant risk of complication during standard ventilation an oxygen supplementation.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

What is claimed is:

1. A method of administering a therapeutic amount of a poly-oxygenated aluminum hydroxide comprising a clathrate containing oxygen gas molecules to a mammal suffering from acute respiratory distress syndrome (ARDS).

2. The method as specified in claim 1 wherein the administration increases recovery time of the mammal to fight the ARDS.

3. The method as specified in claim 1 wherein the administration increases P_{A}O₂ in the mammal.

4. The method as specified in claim 3 wherein the administration increases a P_{A}O₂/FiO₂ ratio in the mammal.

5. The method as specified in claim 1 wherein the administration increases oxygen saturation (sO₂) in the mammal.

6. The method as specified in claim 4 wherein the administration increases oxygen saturation (sO₂) in the mammal.

7. The method as specified in claim 1 wherein the administration increases PO₂ in the mammal.

8. The method as specified in claim 6 wherein the administration increases PO₂ in the mammal.

9. The method as specified in claim 1 wherein the administration reduces amounts of lactate in the mammal

10. The method as specified in claim 8 wherein the administration reduces amounts of lactate in the mammal.

11. The method as specified in claim 1 wherein the administration reduces pH in the mammal.

12. The method as specified in claim 10 wherein the administration reduces pH in the mammal.
